(19) United States
(12) Patent Application Publication
(10) Pub. No.: US 2025/0050084 A1
(43) Pub. Date: Feb. 13, 2025

(54) Applicator and Method of Making Same

(71) Applicant: Solventum Intellectual Properties Company, Maplewood, MN (US)

(72) Inventors: Vinod P. Menon, Woodbury, MN (US); Max A. Kruziki, New Brighton, MN (US); Assumpta A.G. Bennaars-Eiden, Andover, MN (US)

(21) Appl. No.: 18/721,085

(22) PCT Filed: Dec. 19, 2022
(86) PCT No.: PCT/IB2022/062475

§ 371 Date: Jun. 17, 2024

Related U.S. Application Data
(60) Provisional application No. 63/265,724, filed on Dec. 20, 2021.

(51) Int. Cl. A61M 35/00

(52) U.S. Cl. CPC A61M 35/006

(57) ABSTRACT

A liquid applicator for applying a liquid to a surface. The applicator includes a hollow body comprising a wall defining an internal chamber having a closed end and an open end. The applicator further includes a felted polyurethane foam coupled to the open end of the hollow body and an alcohol-based antiseptic solution dispersed within the polyurethane foam. The polyurethane foam has a flatwise tensile strength of at least 1.0 lb^{f}*in/(in² of foam).

### FIELD

The present disclosure relates to an applicator for applying a liquid to a surface, e.g., a surgical prep applicator.

### BRIEF DESCRIPTION OF THE DRAWINGS

FIG. 1 illustrates a side view of a liquid applicator according to some embodiments of the present disclosure.

FIG. 2 illustrates the results of tensile testing of foams in accordance with some embodiments of the present disclosure and comparative foams.

FIG. 3 illustrates the results of tensile testing of felted foams as a function of felting temperature.

### DETAILED DESCRIPTION

It is a standard practice in the industrialized world to disinfect the skin prior to any invasive procedure such as surgery, catheterization, or needle puncture to reduce the risk of infection. These products are often referred to as skin preps or simply “preps.” Antiseptic preparation of patient's skin for such procedures conventionally includes a 30 second-10 minute scrubbing of the affected area with an antiseptic soap solution. These solutions are often applied with foam sponge tipped applicators. The foam sponges are often saturated by soaking them in open pans of solution or with solution from a reservoir contained in a handle fluidically coupled to the foam sponge. The antiseptic solutions are often alcohol-based solutions that tend to cause swelling of the types of foam sponges typically employed (e.g., open-celled polyester or polyether polyurethane foam) (in contrast to water based solutions that are considerably more common in industrial applications for foam sponges, and which do not cause swelling of such foam sponges). Specifically, compressed (or felted) polyurethane foams can reversibly swell in alcohol-based solutions by about 10-15%. This swelling is not expected to compromise the mechanical stability of the foam.

Felting refers to a post-foam generation process where a foam is permanently compressed by placement of the foam between two heated platens and compressing the foam until the compression is permanent. Generally, it has been understood that the felting process yields increased capillarity, which will allow the foam to wick fluid better than an unfelted foam of identical foam composition. However, it was discovered that conventional, commercially available felted foams exhibit irreversible, excessive swelling leading to foam debonding during the above-described scrubbing process involving alcohol-based solutions. More specifically, it was discovered that vigorous scrubbing of the alcohol-based solution soaked, foam-tipped applicator during use on skin can cause irreversible decompression of the felted foam resulting in swelling by as much as 100%. This decompressed foam loses its ability to scrub the skin efficiently as it bunches and rolls during scrubbing. In addition, scrubbing during application with the swollen foam compromises the bond of the foam to the applicator (often referred to as delamination).

Consequently, foam articles and related methods that yield improved dimensional stability and that are capable of consistently avoiding delamination during vigorous scrubbing (even when soaked with an alcohol-based solution) are desirable.

As used herein, the singular forms “a”, “an”, and “the” include plural referents unless the content clearly dictates otherwise. As used in this specification and the appended embodiments, the term “or” is generally employed in its sense including “and/or” unless the content clearly dictates otherwise.

As used herein, the recitation of numerical ranges by endpoints includes all numbers subsumed within that range (e.g. 1 to 5 includes 1, 1.5, 2, 2.75, 3, 3.8, 4, and 5).

Unless otherwise indicated, all numbers expressing quantities or ingredients, measurement of properties and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

In some embodiments, the present disclosure relates to a felted foam pad for applying liquid to a surface. Generally, it has been discovered that certain felting process conditions yield foam articles that exhibit improved dimensional stability and resistance to delamination from an applicator handle during vigorous scrubbing. More specifically, a particular thermal window for felting polyurethane foams has been identified that greatly improves the dimensional stability of foams soaked with alcohol-based solutions. The improved felted foam pad does not exhibit excessive swelling and stochastic debonding of the foam pad from the applicator during the scrubbing protocol.

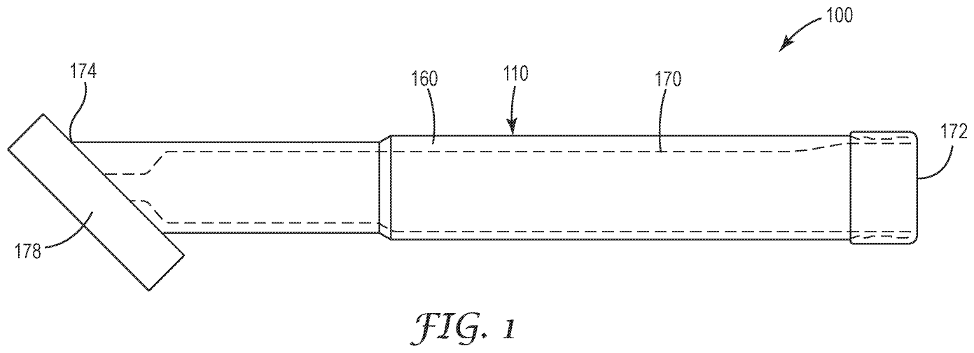

FIG. 1 illustrates a liquid applicator 100 in accordance with some embodiments of the present disclosure. As shown, liquid applicator 100 may include an elongate hollow body 110 (which may serve as a handle for the user) that includes a wall 160. Wall 160 may define chamber 170 having a closed end 172 and an open end 174. Closed end 172 may be sealed in any of a variety of known ways to inhibit or prevent fluid contained within the chamber from escaping through the closed end. For example, a cap, which may be press-fit, screwed, or otherwise attached, may seal the chamber forming closed end 172. The open end 174 may include one or more orifices that permit fluid to flow out of the chamber 170. A pad 178 may be fluidically coupled to the open end 174 such that fluid flowing through the orifice directly or indirectly contacts the pad 178. The pad 178 may be coupled to the hollow body (directly or indirectly) by a conventional fastening mechanism (e.g., adhesives or ultrasonic welding). It is to be appreciated that while the present disclosure has been described with respect to a particular applicator 100, the articles and methods of the present disclosure may be employed with any applicator in which a foam pad is coupled to a handle (which may or may not be hollow) for facilitating scrubbing of a surface (e.g., skin) with the foam pad capable of having an antiseptic solution retained therein.

In some embodiments, an alcohol-based (i.e., a solution having alcohol(s) present in an amount of at least 50 wt. %, based on the total weight of the solution) antiseptic solution can be contained within the chamber 170. Examples of suitable antiseptic solutions include those described in U.S. Pat. No. 4,584,192 and those described in U.S. Pat. No. 4,542,012. Other useful fluids include antiseptic preparations, e.g., iodophoric skin tinctures, such as “Duraprep™ Surgical Solution,” commercially available from 3M. In some embodiments, the antiseptic solution can include an antimicrobial agent such as iodine, an iodine complex (e.g., iodophors), chlorhexidine, chlorhexidine salts (e.g., chlorhexidine digluconate and chlorhexidine diacetate), or combinations thereof. Other exemplary antimicrobial agents include C2-C5 lower alkyl alcohols, fatty acid monoesters of glycerin and propylene glycol, polymers that include a (C12-C22) hydrophobe and a quaternary ammonium group, polyquaternary amines (e.g., polyhexamethylene biguanide), quaternary ammonium silanes, silver, silver salts (such as silver chloride), silver oxide and silver sulfadiazine, methyl, ethyl, propyl and butyl parabens, octenidene, peroxides (e.g., hydrogen peroxide and benzoyl peroxide), and the like, as well as combinations thereof.

In some embodiments, the pad 178 can be prepared from a variety of commercially available materials having a wide range of compression set ratios (i.e., density) and porosities. By varying the compression set ratio and porosity of the foam sponge, applicators can be constructed to apply to a variety of antiseptic solution compositions, viscosities, and volumes.

In some embodiments, the pad 178 may include (or be formed of) a polyurethane foam (e.g., an open-celled polyester or polyether polyurethane foam). Polyurethane foams are generally prepared by the reaction of one or more active hydrogen-containing compounds (i.e., polyols) and one or more polyisocyanates, in the presence of a blowing agent such as water, and usually at least one reaction catalyst and foam stabilizer. The cellular polymer structure of polyurethane foam has a skeletal framework of relatively heavy strands forming an outline for the cell structure. The skeletal framework strands are connected by very thin membranes, often called windows, which form the cell walls. In open-celled foams, some of the windows are open or torn in each cell, thus forming an interconnecting network open to fluid flow (liquid or gas). In some embodiments, the foam may be of the material type described in U.S. Pat. No. 6,841,586 or 8,247,466, which are herein incorporated by reference in their entirety.

In some embodiments, the porosity of the pad 178 may be selected such that the pad will release a uniform amount of liquid when it pressed against the surface upon which the liquid is to be dispensed. In some embodiments, for surgical prep applications, the porosity of the pad, prior to felting, may be between about 10 and 150 pores per linear inch or between about 10 and 90 pores per linear inch.

A wide variety of pad shapes are known. Generally, pad 178 may be oval, square, or rectangular in shape and may be uniform (or substantially uniform) in thickness. In some embodiments, the pad 178 may have a thickness of between 0.1 inch and 5 inches or between 0.2 inches and 2 inches. In some embodiments, the pads 178 may have a working surface area (i.e., area of the surface of the pad intended to contact the skin during scrubbing) of between 0.1 square inches and 2 square inches or between 0.25 square inches and 1.5 square inches. These relatively low working surface areas may be particularly useful in circumstances where delamination of the foam pad from an applicator handle are of concern as shear stress increases as a function of surface area (assuming a constant scrubbing force).

As referenced above, felting refers to a post-process where a foam is permanently compressed by, conventionally, placing a sheet between two heated platens and compressing the foam until the compression is permanent. Generally, it is known that the felted foam will have increased capillarity, which will allow the foam to wick fluid better than an unfelted foam of identical foam composition. The compression ratio is calculated as the pre-felting thickness over the post-felting thickness. The compression ratio is generally referred to as a firmness. For example, a foam compressed to one-third of its original thickness is a firmness 3 felt and has a compression ratio of 3. A foam compressed to one-fifth of its original thickness is a firmness 5 felt.

Conventionally, while compressed, the foams are heated to a temperature of from 340 to 380° F. (171 to 193° C.) for from 10 to 60 minutes to achieve felting. The resulting felted foams exhibit excellent dry internal strength and its compression set is difficult to reverse by normal mechanical means. Elevated temperatures and times have been considered generally undesirable as they are not believed to provide additional value and can result in undue thermal degradation. However, it has been discovered that, unexpectedly, the compression of the unfelted foam at a temperature above the conventional temperature range greatly improves felting strength and resistance to decompression of the foam when scrubbing with an alcohol-based antiseptic solution. Specifically, it has been discovered that the compression of the unfelted foam at a temperature of at least 430° F. (at felting temperatures lower than 430° F., the resulting foam does not possess the desired increased internal strength) and no more than 450° F. (at felting temperatures above 450° F., the foam begins to degrade) for at least 1 minute, at least 10 minutes, at least 20 minutes, or at least 30 minutes yields felted foam pads with greatly improved felting strength and resistance to decompression of the foam when scrubbing with an alcohol-based antiseptic solution.

In some embodiments, the foam pads of the present disclosure may be felted by compressing the foam from one-half (½) to one-twentieth ( 1/20) of its initial thickness, or from one-third (⅓) to one-eighth (⅛) of its initial thickness, under the above-described heating conditions. In some embodiments, felted foam pads having a compression ratio between 2 and 4 may be particularly useful as a result of their desirable balance of fluid-handling capabilities and comfort on the skin of a patient during scrubbing.

In some embodiments, the foam pads of the present disclosure may be double-felted. As used herein, double-felted refers to a pad that is subjected to a first felting process at a conventional temperature range (a temperature of from 340 to 380° F. (171 to 193° C.) for from 10 to 60 minutes) and a second felting process at the temperature range of the present disclosure (at least 430° F. and no more than 450° F. for at least 1 minute, at least 10 minutes, at least 20 minutes, or at least 30 minutes). Such double-felting may be accomplished by, for example, subjecting a commercially available felted foam to a felting process that employs the elevated temperature range of the present disclosure.

The internal felting strength, or resistance to reversing the felt, can be described as the force required to pull the two faces of the foam apart in the felted axis direction, also called the flatwise foam tensile strength. As used herein, the test method for determining flatwise foam tensile strength was adapted from ASTM C297 for use with flexible foams:
1. A 1.5×1.5 inch piece of felted foam is adhered between two flat metal t-shaped plates. The plates are adhered such that the felting axis of the foam is perpendicular to the flat surface of the plates.
2. A tensile tester is used to measure the force to pull the plates apart at 12 inch/min until the foam has reached 125% of its felted thickness.
3. Total work, or the integral of the force vs. distance curve, is calculated. The result is divided by the cross-sectional area of the foam sample.
   The total work required to separate the two foam faces is higher for a foam that has a stronger felt at a given initial thickness and felt firmness.

In some embodiments, in accordance with the test methods of the present disclosure, the felted foam pads may have a flatwise tensile strength of at least 0.5 lb^{f}*in/(in² of foam), at least 1.0 lb^{f}*in/(in² of foam), or at least 1.5 lb^{f}*in/(in² of foam).

In some embodiments, the present disclosure may be further directed to methods of making the above-described foam pads. The method may include providing an unfelted or conventionally felted polyurethane foam (e.g., an open-celled polyester or polyether polyurethane foam). The method may then include, if unfelted, compressing the unfelted polyurethane foam to from one-half (½) to one-twentieth ( 1/20) of its initial thickness (e.g., by placing between two platens and applying a pressing force via the platens) The method may then include subjecting the compressed foam to a temperature of at least 430° F. and no more than 450° F. for at least 1 minute, at least 10 minutes, at least 15 minutes, at least 20 minutes, or at least 30 minutes.

In some embodiments, the present disclosure may be further directed to methods of using the above-described applicators 100 to prepare a site on the skin of a mammal (e.g., a surgical site). The method may include introducing the alcohol-based liquid solution into the foam pad 178 (e.g., such that the foam pad 178 is saturated or nearly saturated with the liquid solution) and then contacting (e.g., scrubbing) the site with the foam pad for a period of at least 30 seconds, at least 1 minute, at least 2 minutes, at least 3 minutes, at least 4 minutes, at least 5 minutes, at least 6 minutes, at least 7 minutes, at least 8 minutes, at least 9 minutes, or at least 10 minutes. For purpose of the present application, “scrubbing” refers to any shearing action that the foam pad undergoes while in contact with skin site. For example, “scrubbing” may refer to application of a downward force in a direction normal (or substantially normal) to the skin site while simultaneously moving the pad in a direction parallel (or substantially parallel) to the skin site (e.g., back and forth motion, rotational motion, a combination of back and forth and rotational motion). As discussed above, as a result of the stronger “internal weld” of the felted foams of the present disclosure, during such extended scrubbing, the felted foam pads of the present disclosure resist decompression (or reverse felting) and, therefore, delamination of the felted foam pad from the applicator handle.

The operation of the present disclosure will be further described with regard to the following detailed examples. These examples are offered to further illustrate various embodiments and techniques. It should be understood, however, that many variations and modifications may be made while remaining within the scope of the present disclosure.

### EXAMPLES

Foam with product code FLTZ93MA from FXI Holdings, Inc. (“FXI”, Radnor, PA) was used for all Examples. A method for manufacture of these foams is found in U.S. Pat. No. 6,371,606 assigned to FXI, column 4, line 3 to column 5, line 42. Briefly, the method comprises forming a closed cell polyurethane foam by reacting a polyether polyol with a polyisocyanate in the presence of a catalyst, a blowing agent, a foam stabilizer, and optionally other foaming aids, reticulating the foam through flame treatment, and felting through compression between heated plates.

### Example 1

Felted foam samples from two different runs (foam poured by FXI and felted by UFP Technologies, Inc., Newburyport, MA, “UFP”) were chosen for the experiment. These two foam samples are referred to as untreated foams. Both foam samples were 0.395 inches thick and had a pore density of 93 pores per inch (ppi). The only difference was in the felting, which was conducted with compression ratios or firmness of 3.8 and 4.0.

The foams were felted again (double felted) by lightly compressing them between two stainless steel plates at a pressure of 0.12 pounds per square inch (PSI) and placing them in an oven preheated to 455° F. The foams were heat-treated for 45 minutes. At the end of the experiment, the temperature of the stainless-steel plates was measured using an IR gun and determined to be around 355° F.

The double felted foams were found to decrease in thickness by about 0.025-0.05 inches. This is thought to be due to internal melting and re-sintering of the flattened foam cells. The double felted foams were slightly darker than the untreated foams. After cooling, the double felted foams were attached to 10.5 mL applicators using a hot glue gun. The double felted foams were each loaded with 10.5 mL tinted 70% isopropanol solution and scrubbed for two minutes on a cardboard template using a back-and-forth and side-to-side motion covering a 1″×5″ area. Untreated foams (foams that were not subjected to the second heat treatment) de-felted, rolled, and bunched in under 5 seconds. The double felted foams, on the other hand, held up for the entire two minutes of scrubbing. No bunching or visible de-felting of treated foams was observed, though some rolling occurred. This study was repeated using excised pig skin as the scrubbing substrate and produced the same results. The untreated foam felted at 4.0 swelled by about 150%, while the double felted foam swelled by only 11%.

### Example 2

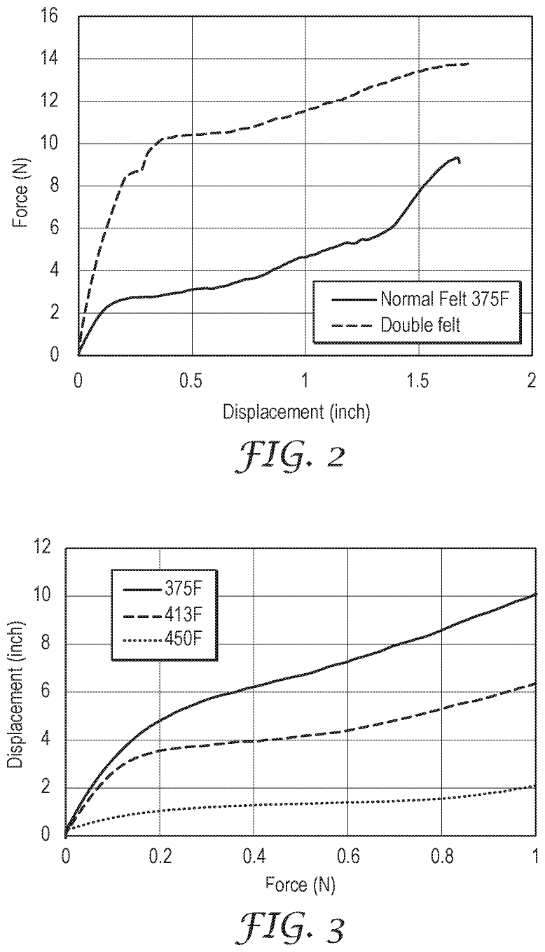

Tensile testing of treated and double felted foams (0.75″ diameter circular pieces) in the z-direction (transverse or short axis of the foam) showed that the double felted foam samples had substantial improvement in both the stiffness (slope of the initial deformation) and yield strength (abrupt change in slope) as shown in FIG. 2. These data show that untreated felted foam was substantially improved in internal strength by a thermal post-treatment under pressure.

### Example 3

A single foam was poured by FXI and felted by FXI at temperatures of 375° F., 413° F., and 450° F. for 10 minutes. Tensile testing of the foams (0.75″ diameter circular pieces) in the z-direction (transverse or short axis of the foam) shows substantial improvement in both the stiffness (slope of the initial deformation) and yield strength (abrupt change in slope) as shown in FIG. 3. These data show that the internal strength of the foam increases proportionally with felting temperature.

### Example 4

Foam was poured by FXI and samples were felted at 375° F. by UFP or at 450° F. degrees by FXI (thickness 0.38-0.40″, firmness 3.8-4.0, porosity 90-100 ppi). Tensile testing of the foam in the z-direction was carried out according to ASTM C297 with some modifications. In short, foam was cut to 1.375×1.375 inch squares, adhered between two flat metal plates with double-sided adhesive tape, tensile testing in the felting axis was performed to 0.5 inch displacement at a speed of 12 inch/minute, and values were reported as Work (area under the force vs. displacement curve) per area of foam. Foam was either tested dry or saturated with 70% isopropanol. Results are presented in Table 1. These data show that increased foam strength from high temperature felting is maintained after the foam is exposed to isopropanol.

TABLE 1
Flatwise Tensile Test Results Of Dry And Wetted Foams

Work to 0.5 inch
(lbf*in/in²)

| Felt | | 70% IPA |
|---|---|---|
| temperature | Dry | Saturated |
| 450° F. | 1.8 | 0.65 |
| 375° F. | 0.39 | 0.14 |

### Example 5

Foam was poured by FXI Corp. and felted at 375° F. by UFP or at 450° F. by FXI. Foam samples (1.375×1.375 inch squares, thickness 0.38-0.40″, firmness 3.8-4.0, porosity 90-100 ppi) were attached to plastic applicators using hot plate welding. The attached foams were filled with 10.5 mL of isopropanol and scrubbed on cardboard substrates for up to 3 minutes. At the end of scrubbing the foam thickness was measured, compared to the pre-scrub foam thickness, and the percentage increase in thickness was calculated. If the foam separated (delaminated) from the applicator prior to 3 minutes, the time was recorded. Results are provided in Table 2.

TABLE 2
Effect Of Felt Temperature On Foam Swelling
And Delamination Properties

| Felt Temperature | Increase in foam | Time to foam delamination |
|---|---|---|
| (° F.) | swelling (%) | (seconds) |
| 375 | 52 | 106 |
| 450 | 10 | No delamination |
| | | at 180 seconds |

This data shows that increased felting temperature led to less foam swelling and fewer foam delaminations from the applicators.

### Example 6

A single foam was poured by FXI and felted by FXI at varying temperatures and times. The foam had thickness 0.38″, firmness 4.0, and porosity 90-100 ppi. Tensile testing of the foam in the z-direction was carried out according to ASTM C297. In short, foam was cut to 1.375×1.375 inch squares, adhered between two flat metal plates with double-sided adhesive tape, tensile testing in the felting axis was performed to 0.5 inch displacement at a speed of 12 inch/minute, and values were reported as Work (area under the force vs. displacement curve) per area of foam. Results are provided in Table 3.

TABLE 3
Tensile Properties Of Foams Felted At
Three Times And At Three Temperatures

| Felt Temperature | Felt Time | Work to 0.5 inch |
|---|---|---|
| (° F.) | (minutes) | (lbf*in/in²) |
| 375 | 10 | 0.39 |
| 375 | 20 | 0.51 |
| 375 | 30 | 1.03 |
| 413 | 10 | 1.04 |
| 413 | 20 | 1.17 |
| 413 | 30 | 1.38 |
| 450 | 10 | 1.68 |
| 450 | 20 | 1.63 |
| 450 | 30 | 1.68 |

These data show that for a 10 minute felt time, approximately 413° F. felt temperature is preferred for increased scrubbing capacity. At longer felt times, lower temperatures can achieve a similar foam internal strength as a 10 minute higher temperature felt.

### Example 7

A designed experiment was done to evaluate the performance of foams felted at various temperatures and times (FXI Corp). Conditions are indicated in Table 4 under Foam Type. For example, the first foam was felted with firmness of 3.8, had thickness 0.395 inch, the felting temperature was 375° F., and the felting time was 10 minutes.

The foams were attached to applicators and used to apply 10.5 mL of 70% isopropanol topically to a 1×5 inch area of ex-vivo pig skin by scrubbing back and forth and side to side vigorously for a total of 2 minutes. After completion of the scrub time, the foams were evaluated for swelling, bunching and rolling.

TABLE 4
Percentage Swelling After Scrubbing
Challenge Of Foams Felted Under The Conditions Indicated

| | Percent swell after |
|---|---|
| Foam Type | 2 minute scrub |
| 3.8 0.395 375° F. 10 min | 42 |
| 4.0 0.395 375° F. 10 min | 30 |
| 4.0 0.380 375° F. 20 min | 30 |
| 4.0 0.380 375° F. 30 min | 15 |
| 4.0 0.380 413° F. 10 min | 16 |
| 4.0 0.380 413° F. 20 min | 16 |
| 4.0 0.380 413° F. 30 min | 17 |
| 4.0 0.380 450° F. 10 min | 18 |
| 3.8 0.395 double felt | 11 |
| 4.0 0.395 double felt | 11 |

Increasing felting temperature and longer times at lower temperatures resulted in foam with minimal swelling (below 20%) for foams felted at 375° F. for 30 minutes, 413° F. for 10-30 minutes and 450° F. for 10 minutes. Results are provided in Table 4. The double felted foams showed the least swelling of approximately 12%.

The data for foam bunching and rolling followed the same trend as the swell data. Foams that were felted at higher temperatures lasted a full 120 seconds without bunching or rolling. At lower temperatures (e.g., 375° F.), as the felt time increased, the time to bunch and roll was extended from 20 seconds for 375° F./10 minutes to just over 100 seconds for 375° F./30 minutes. Increasing the temperature to 413° F. with a shorter felt time of 10 minutes resulted in a foam that bunched and rolled in approximately 60 seconds. The double felted foams did not bunch and roll at 120 seconds. Results are provided in Table 5.

TABLE 5
Time To Excessive Rolling During Scrubbing
Challenge Of Foams Felted Under The Conditions Indicated

| Foam Type | Time to excessive rolling (secs) |
|---|---|
| 3.8 0.395 375° F. 10 min | 15 |
| 4.0 0.395 375° F. 10 min | 30 |
| 4.0 0.380 375° F. 20 min | 60 |
| 4.0 0.380 375° F. 30 min | 106 |
| 4.0 0.380 413° F. 10 min | 47 |
| 4.0 0.380 413° F. 20 min | 120 |
| 4.0 0.380 413° F. 30 min | 120 |
| 4.0 0.380 450° F. 10 min | 120 |
| 3.8 0.395 double felt | 120 |
| 4.0 0.395 double felt | 120 |

1. A liquid applicator for applying a liquid to a surface, the liquid applicator comprising:
a hollow body comprising a wall defining an internal chamber having a closed end and an open end;
a felted polyurethane foam coupled to the open end of the hollow body; and
an alcohol-based antiseptic solution dispersed within the polyurethane foam;
wherein the polyurethane foam has a flatwise tensile strength of at least 1.0 lb^{f}*in/(in² of foam).

2. The liquid applicator of claim 1, wherein the felted polyurethane foam is selected from the group consisting of an open-celled polyester and a polyether polyurethane foam.

3. The liquid applicator of claim 1, wherein the porosity of the pad, prior to felting, is between 10 pores per linear inch and 150 pores per linear inch.

4. The liquid applicator of claim 1, wherein the antiseptic solution is selected from the group consisting of iodine, an iodine complex, chlorhexidine, a chlorhexidine salt, a C2-C5 lower alkyl alcohol, a fatty acid monoester of glycerin and propylene glycol, a polymer that includes a C12-C22 hydrophobe and a quaternary ammonium group, a polyquaternary amine, a quaternary ammonium silane, silver, a silver salt, a silver oxide, a silver sulfadiazine, methyl, ethyl, propyl or butyl parabens, octenidene, and a peroxide.

5. The liquid applicator of claim 1, wherein the antiseptic solution is selected from the group consisting of iodine, an iodine complex, chlorhexidine, and chlorhexidine salt.

6. A method of preparing a site on skin of a patient, the method comprising:
providing a liquid applicator, comprising;
a hollow body comprising a wall defining an internal chamber having a closed end and an open end;
a felted polyurethane foam coupled to the open end of the hollow body; and
an alcohol-based antiseptic solution dispersed within the polyurethane foam,
wherein the polyurethane foam has a flatwise tensile strength of at least 1.0 lb^{f}*in/(in² of foam); and
scrubbing the site with the felted polyurethane foam for a period of at least one minute.

7. The method of claim 6, wherein the site on the skin of the patient comprises a surgical site.

8. A method of making a felted polyurethane foam, the method comprising:
providing a polyurethane foam;
compressing the foam from one-half (½) to one-twentieth ( 1/20) of its initial thickness;
subjecting the polyurethane foam to a temperature of no less than 430° F. and no more than 450° F. for at least 5 minutes.

9. The method of claim 8, wherein the polyurethane foam is selected from the group consisting of an open-celled polyester and a polyether polyurethane foam.

10. The method of claim 6, further comprising:
coupling the felted polyurethane foam to a handle:
introducing an alcohol-based antiseptic solution into the felted polyurethane foam;
scrubbing the site with the felted polyurethane foam for a period of at least 30 seconds

11. The method of claim 10, wherein the antiseptic solution is selected from the group consisting of iodine, an iodine complex, chlorhexidine, a chlorhexidine salt, a C2-C5 lower alkyl alcohol, a fatty acid monoester of glycerin and propylene glycol, a polymer that includes a C12-C22 hydrophobe and a quaternary ammonium group, a polyquaternary amine, a quaternary ammonium silane, silver, a silver salt, a silver oxide, a silver sulfadiazine, methyl, ethyl, propyl or butyl parabens, octenidene, and a peroxide.
